(19) United States
(12) Patent Application Publication
(10) Pub. No.: US 2013/0108557 A1
(43) Pub. Date: May 2, 2013

(54) COSMETIC FOAM

(71) Applicants: Albert Zorko Abram, Wantirna (AU); Iulian Goldstein, Ferntree Gully (AU)

(72) Inventors: Albert Zorko Abram, Wantirna (AU); Iulian Goldstein, Ferntree Gully (AU)

(21) Appl. No.: 13/498,346

(22) PCT Filed: Sep. 28, 2010
(86) PCT No.: PCT/AU10/01269

§ 371 Date: Jul. 24, 2012

Related U.S. Application Data
(60) Provisional application No. 61/247,073, filed on Sep. 30, 2009.

(51) Int. Cl. A61K 9/46; A61Q 19/00; A61K 47/46; A61K 8/97

(52) U.S. Cl. CPC A61K 9/0007; A61K 8/97; A61Q 19/00; A61K 47/46
USPC 424/45

(57) ABSTRACT

The present invention provides aerosol foams comprising Aloe vera. In particular, the foams comprise (a) Aloe vera, (b) a surfactant and (c) a propellant. The foams may further comprise one or more of the group consisting of an α-hydroxy acid, a β-hydroxy acid, a humectant, a thickening agent, a dermatologically acceptable excipient, and mixtures thereof.

### FIELD

The present invention relates to topical aerosol foams comprising Aloe vera.

### BACKGROUND

As described in Martindale, The Complete Drug Reference, 34^{th} Edition, Sean C Sweetman Ed., Pharmaceutical Press (2005), Aloe vera gel is a mucilaginous preparation obtained from the leaves of Aloe vera (Aloe barbadensis), which is widely used in cosmetics for its moisturising and revitalising properties.

U.S. Pat. No. 5,002,680 (Schmidt et al) describes an aerosol foam for moisturising the skin. The foam comprises 88% to 97% of a concentrate and 3% to 12% of a propellant, wherein the foam has a pH in the range from about 5 to about 6. The concentrate comprises from 3% to 20% of a mild non-soap anionic or amphoteric surfactant, 0.05% to 5% of a polymeric skin feel aid, 10% to 60% of a moisturiser, and water. The concentrate may also comprise Aloe vera gel (0.5% to 5%).

U.S. Pat. Nos. 5,716,625 and 6,139,850 (Hahn and Thueson) describe topical preparations comprising (i) an irritating active ingredient such as α- and β-hydroxy acids, retinoids and peroxides, and (ii) a divalent strontium cation. The divalent cation is able to reduce the irritation attributable to the active ingredient. The composition may further comprise Aloe vera.

U.S. Pat. No. 7,252,846 (Dinno) describes a therapeutic composition suitable for the treatment of skin conditions such as psoriasis, eczema, ichthyosis, pruritis, dryness and dermatitis. The composition contains a number of natural and synthetic components, namely mineral oil, zinc oxide, quarternium-18 hectorite, magnesium hydroxide, cod liver oil, gum karaya powder, propylene carbonate, tea tree oil, aloe powder and lanolin.

U.S. Pat. No. 7,262,224 (Chong) describes a cosmetic composition for rejuvenating the appearance of the skin. The composition is preferably in the form of a lotion or cream and comprises vitamins A, B, C, D and E, along with Aloe vera.

U.S. Pat. No. 7,357,950 (Mazzio and Soliman) is directed to a topical skin care composition suitable for treating dry skin or dyshidrosis. The composition comprises a) black walnut, wormwood, turmeric and garlic, b) two or more antibacterial/anti-inflammatory herbs selected from the group consisting of St. John's wort, licorice, chamomile, clove, nutmeg, ginger and myrrh, c) Aloe vera and d) niacin, niacinamide and nicotinic acid.

α-Hydroxy acids such as glycolic acid or lactic acid are known for their ability to exfoliate the skin and, in doing so, rejuvenate it. β-Hydroxy acids such as salicylic acid are similarly able to act as exfoliants in topical preparations.

U.S. Pat. No. 4,363,815 (Yu & Van Scott) describe therapeutic compositions comprising α- or β-hydroxy acids, suitable for the treatment of dry skin, psoriasis, acne, dandruff, Darier's disease, palmar and plantar hyperkeratosis, ichthyosis, acne, eczema, pruritic, warts, lichen simplex chronicus, keratoses and herpes, for example. The patent describes that the α- and β-hydroxy acids may be formulated in a solution, lotion, gel, shampoo, spray, stick, powder, cream or ointment.

There remains a need in the art for topical preparations that are cosmetically elegant and are able to moisturize, revitalize and rejuvenate the skin. The present invention addresses these unmet needs.

### SUMMARY OF THE INVENTION

According to an embodiment, the present invention provides a topical aerosol foam composition comprising: (a) Aloe vera, (b) a surfactant and (c) a propellant.

According to a further embodiment, the invention provides a method of moisturising the skin, the method comprising topically administering to a subject in need thereof, a cosmetically effective amount of an aerosol foam composition comprising: (a) Aloe vera, (b) a surfactant and (c) a propellant.

According to another embodiment, the invention relates to the use of an aerosol foam composition comprising: (a) Aloe vera, (b) a surfactant and (c) a propellant, in the manufacture of a cosmetic preparation for moisturizing the skin.

According to yet another embodiment, the present invention relates to the use of an aerosol foam composition comprising: (a) Aloe vera, (b) a surfactant and (c) a propellant, for moisturizing the skin.

In an embodiment, the present invention provides a topical aerosol foam composition comprising: (a) Aloe vera, (b) a pharmaceutically active agent, (c) a surfactant and (d) a propellant.

In a further embodiment, the present invention provides a method of treating a disease, disorder or condition of the skin, the method comprising topically administering to a subject in need thereof, a therapeutically effective amount of an aerosol foam composition comprising: (a) Aloe vera, (b) a pharmaceutically active agent, (c) a surfactant and (d) a propellant.

In another embodiment, the invention relates to the use of an aerosol foam composition comprising: (a) Aloe vera, (b) a pharmaceutically active agent, (c) a surfactant and (d) a propellant, in the manufacture of a medicament for the treatment of a disease, disorder or condition of the skin.

In yet another embodiment, the invention relates to the use of an aerosol foam composition comprising: (a) Aloe vera, (b) a pharmaceutically active agent, (c) a surfactant and (d) a propellant, for the treatment of a disease, disorder or condition of the skin.

### DETAILED DESCRIPTION OF THE INVENTION

The present invention provides a topical aerosol foam composition comprising: (a) Aloe vera, and (b) a surfactant, suitably in a pressurized container containing a propellant.

According to an embodiment, the topical aerosol foam breaks easily on the skin with application of shear.

### Aloe Vera

In an embodiment, the Aloe vera used herein is Aloe vera gel, also known as Aloe barbadensis leaf juice. Aloe vera gel is commercially available as Aloe barbadensis leaf juice in an unconcentrated (native) form and may be used in the present compositions.

Aloe vera gel is also commercially available as 2×, 5×, 10×, 20×, 40× and 200× concentrates. Where a concentrate is used in preparation of the present compositions, the concentrate is diluted with water to achieve the equivalent of 100% Aloe vera gel (juice), that is, to mimic Aloe barbadensis in its unconcentrated (native) form. Accordingly, in this embodiment, the composition further comprises water in an amount from about 40% to about 95% by weight, from about 40% to about 70% by weight, from about 70% to about 95% by weight, or from about 80% to about 95% by weight.

Suitably, the Aloe vera gel concentrate is present in an amount from about 0.1% to about 50% by weight. As will be appreciated by the person skilled in the art, the quantity of Aloe vera concentrate used in the compositions of the invention will vary according to concentrate used (i.e. 2×, 5×, 10×, 20×, 40× or 200×).

Where a 2× concentrate is used in the compositions of the invention, the Aloe vera concentrate will suitably be present in an amount from about 10% to about 50% by weight. Where a 5× concentrate is used, the concentrate will suitably be present in an amount from about 4% to about 20% by weight. Where a 10× concentrate is used, the concentrate will suitably be present in an amount from about 2% to about 10% by weight. Where a 20× concentrate is used, the concentrate will suitably be present in an amount from about 1% to about 5% by weight. Where a 40× concentrate is used, the concentrate will suitably be present in an amount from about 0.5% to about 2.5% by weight. Where a 200× concentrate is used, the Aloe vera concentrate is suitably present in an amount from about 0.1% to about 0.5% by weight.

In an embodiment, the aerosol foam compositions of the invention comprise Aloe vera gel as a 40× concentrate in an amount from about 0.5% to about 2.5% by weight, water in an amount from about 70% to about 95% by weight, a surfactant and a propellant.

In an alternative embodiment, the aerosol foam compositions of the invention comprise Aloe vera gel as a 200× concentrate in an amount from about 0.1% to about 0.5% by weight, water in an amount from about 80% to about 95% by weight, a surfactant and a propellant.

### Surfactant

In an embodiment, the surfactant is a non-ionic surfactant.

Exemplary non-ionic surfactants include, but are not limited to, ethoxylated carboxylic acids such as PEG-8 stearate, PEG-12 stearate, PEG-20 stearate, PEG-30 stearate, PEG-40 stearate, PEG-50 stearate, PEG-100 stearate, PEG-12 distearate, PEG-32 distearate, PEG-150 distearate and PEG-10 oleate; ethoxylated glycerides such as PEG-35 castor oil, PEG-40 hydrogenated castor oil and PEG-60 hydrogenated castor oil; polyhydric alcohol esters and ethers, such as methyl gluceth-20 sesquistearate; ethoxylated sorbitan esters such as polysorbate 20, polysorbate 40, polysorbate 60 and polysorbate 80; ethoxylated alcohols such as ceteareth-6, ceteareth-12, ceteareth-20, ceteareth-25, ceteth-10, ceteth-20, laureth-4, laureth-5, laureth-9, laureth-10, laureth-12, laureth-15, laureth-20, laureth-23, oleth-10, oleth-20, steareth-10, steareth-20 and steareth-100; ethoxylated lanolin such as PEG-20 lanolin, PEG-30 lanolin, PEG-75 lanolin, PEG-100 lanolin and PEG-150 lanolin; ethoxylated polysiloxanes such as dimethicone copolyol; propoxlated POE ethers such as poloxamer 124, poloxamer 188, poloxamer 237, poloxamer 338 and poloxamer 407; and alkylpolyglucosides such as caprylyl glucoside, decyl glucoside, lauryl glucoside, coco-glucoside, cetearyl glucoside and isostearyl glucoside, and mixtures thereof.

In an embodiment, the non-ionic surfactant is an alkylpolyglucoside. Suitably, the alkylpolyglucoside is selected from the group consisting of caprylyl glucoside, decyl glucoside, lauryl glucoside, coco-glucoside, cetearyl glucoside and isostearyl glucoside, and mixtures thereof. In a particular embodiment, the alkylpolyglucoside is decyl polyglucoside.

In another embodiment, the non-ionic surfactant is an ethoxylated alcohol. Suitably, the ethoxylated alcohol is selected from the group consisting of ceteareth-6, ceteareth-12, ceteareth-20, ceteareth-25, ceteth-10, ceteth-20, laureth-4, laureth-5, laureth-9, laureth-10, laureth-12, laureth-15, laureth-20, laureth-23, oleth-10, oleth-20, steareth-10, steareth-20 and steareth-100. In a particular embodiment, the ethoxylated alcohol is ceteareth-20.

In an embodiment, the surfactant is present in an amount from about 0.1% to about 5% by weight. In a further embodiment, the surfactant is present in an amount from about 0.5% to about 1% by weight.

### Propellant

The propellant may be any suitable liquefied gas, such as a hydrocarbon, a chlorofluorocarbon, dimethyl ether, a hydrofluorocarbon and mixtures thereof. Other suitable propellants include compressed gases such as nitrogen, carbon dioxide, nitrous oxide and air. In an embodiment, the propellant is a hydrocarbon propellant, such as a mixture of hydrocarbons. Suitably, the hydrocarbon propellant comprises a mixture of propane, n-butane and isobutane. One of skill in the art would appreciate that other combinations of propellant are useful in the present invention.

Suitably, the propellant is present in an amount from about 2.5% to about 10% by weight, such as about 5% by weight.

### Thickening Agent

According to an embodiment, the compositions of the invention further comprise a thickening agent.

Exemplary thickening agents include, but are not limited to, agar; acrylic acid polymers and copolymers including Acritamers, Carbomers and Pemulens; alginates and salts and derivatives thereof including the ammonium salt, sodium salt and potassium salt and propylene glycol alginate; carageenan including kappa (sodium salt), iota (sodium salt) and lambda; chitosan; cellulose derivatives including hydroxyethyl cellulose, hydroxyethyl methylcellulose, hydroxypropyl cellulose, hydroxypropyl methylcellulose, methylcellulose and sodium carboxymethylcellulose; dextrin; gelatin; gum including acacia, ceratonia, guar, tragacanth and xanthan gum; gum derivatives including hydroxypropyl guar and guar hydroxypropyltrimonium chloride; maltodextrin; pectin; polycarbophil; polydextrose; polyethylene glycol (PEG) including PEG 200, PEG 300, PEG 400, PEG 540, PEG 600, PEG 900, PEG 1000, PEG 1450, PEG 1500, PEG 1540, PEG 2000, PEG 3000, PEG 3350, PEG 4000, PEG 4600, PEG 6000, PEG 8000, PEG 20000 and PEG 35000; polyethylene oxide; poly(methyl vinyl ether/maleic anhydride) and copolymers and derivatives thereof; polyvinyl alcohol; polyvinylpyrrolidone (PVP) including PVP K12, K15, K17, K25, K30, K60, K90 and K120; pregelatinized starch; hyaluronan including hyaluronic acid and sodium hyaluronate; sulfobutylether beta-cyclodextrin and trehalose and mixtures thereof.

In an embodiment, the thickening agent is selected from the group consisting of propylene glycol alginate; cellulose derivatives including hydroxyethyl cellulose, hydroxyethyl methylcellulose, hydroxypropyl cellulose, hydroxypropyl methylcellulose, methylcellulose and sodium carboxymethylcellulose; and polyvinylpyrrolidone (PVP) including PVP K12, K15, K17, K25, K30, K60, K90 and K120, and mixtures thereof. The molecular weight of PVP ranges from approximately 2500 for K12 to 3,000,000 for K120.

Suitably, the thickening agent is a cellulose derivative. In an embodiment, the cellulose derivative is selected from the group consisting of hydroxyethyl cellulose, hydroxyethyl methylcellulose, hydroxypropyl cellulose, hydroxypropyl methylcellulose, methylcellulose and sodium carboxymethylcellulose, and mixtures thereof.

In another embodiment, the thickening agent is xanthan gum.

According to a further embodiment, the thickening agent is present in an amount from about 0.01% to about 2% by weight. Suitably, the thickening agent is present in an amount from about 0.1% to about 1% by weight, such as about 0.1%, 0.2%, 0.3%, 0.4%, 0.5%, 0.6%, 0.7%, 0.8%, 0.9% or 1% by weight, for example.

### Humectant

In an embodiment, the present compositions further comprise a humectant.

Exemplary humectants include, but are not limited to, alcohols such as 1,4-butanediol, 2,3-butanediol, butylene glycol, ethylene glycol, hexanediol, 1,2-hexanediol, hexylene glycol, pentylene glycol, propanediol, propylene glycol and panthenol; amides such as urea; biological products and their derivatives such as honey and hydrogenated honey; carbohydrates such as trehalose and xylose; ethers such as polyglyceryl sorbitol; organic salts such as potassium cocoyl PCA, potassium lactate, potassium lauroyl PCA, potassium PCA, sodium glucuronate, sodium lactate, sodium malate and sodium PCA; and polyols such as diglycerin, erythritol, fructose, glucose, glycerin, 1,2,6-hexanetriol, maltitol, maltose, mannitol, mannose, sorbitol, sucrose, tripropylene glycol and xylitol, and mixtures thereof.

In another embodiment, the humectant is a mixture of at least two humectants. In another embodiment, the humectant is a mixture of three of more. In yet another embodiment, the humectant is a mixture of at least 4.

Suitably, when there is more than one humectant, the humectant consists of one or more of the group consisting of glycerin, propylene glycol, sodium PCA and D-panthenol. In an embodiment, the humectant is a mixture of glycerin, propylene glycol, sodium PCA and D-panthenol. In this embodiment, the glycerin, propylene glycol, sodium PCA and D-panthenol are each present in an amount from about 0.25% to about 2% by weight. In an embodiment, the ratio of glycerin, propylene glycol, sodium PCA and D-panthenol is about 1:1:1:1. In another embodiment, the ratio of glycerin, propylene glycol, sodium PCA and D-panthenol is about 1:1:1.4:1.

In an embodiment, the humectant is present in an amount from about 1% to about 20% by weight. Suitably, the humectant is present in an amount from about 2% to about 8% by weight, such as about 2%, 3%, 4%, 5%, 6%, 7% or 8% by weight.

### Lower Alcohol

According to an embodiment, the aerosol foam composition comprises less than 15% by weight of a C₁ to C₆ alcohol (i.e. lower alcohol). Suitable lower alcohols include but are not limited to ethanol, propanol, isopropanol, n-butyl alcohol, or t-butyl alcohol. In an embodiment, the C₁ to C₆ alcohol is a mixture of C₁ to C₆ alcohols. In a further embodiment, the aerosol foam composition comprises from about 1% to about 5% C₁ to C₆ alcohol by weight. In another embodiment, the compositions are substantially devoid of C₁ to C₆ alcohol and, suitably, the compositions are devoid of C₁ to C₆ alcohol.

### Pharmaceutically Active Agent

According to yet another embodiment, the aerosol foam compositions further comprise a pharmaceutically active agent. In an embodiment, the pharmaceutically active agent is selected from the group consisting of a keratolytic agent, a corticosteroid, an antihistamine, a wound healing agent, an antiseptic, an antibacterial agent, an antifungal agent, a retinoid, a vitamin D analogue, a skin conditioning agent, a nutritional agent, a sunscreen, and mixtures thereof.

In a further embodiment, the pharmaceutically active agent is selected from the group consisting of a keratolytic agent, an antibacterial agent, and a skin conditioning agent.

In a particular embodiment, the pharmaceutically active agent is a keratolytic agent. In an alternative embodiment, the pharmaceutically active agent is an antibacterial agent. In a further embodiment, the pharmaceutically active agent is a skin conditioning agent.

In an embodiment, the pharmaceutically active agent is present in an amount from about 0.001% to about 30% by weight, depending on the nature of the active agent or combination of active agents.

Exemplary keratolytic agents include, but are not limited to, urea, propylene glycol and art recognized keratolytic acids.

In an embodiment, the keratolytic agent is at least one acid. In a further embodiment, the acid is selected from the group consisting of an α-hydroxy acid and a β-hydroxy acid, and mixtures thereof. In one embodiment the acid is at least one α-hydroxy acid. In an alternative embodiment, the acid is at least one β-hydroxy acid.

Exemplary α-hydroxy acids include, but are not limited to, glycolic acid, lactic acid, citric acid, malic acid, α-hydroxyethanoic acid, α-hydroxyoctanoic acid, α-hydroxycaprylic acid, mixed fruit acid, triple fruit acid, tri-alpha hydroxy fruit acids, sugar cane extract, alpha hydroxy and botanical complex, L-alpha hydroxy acid and glycomer in crosslinked fatty acids alpha nutrium, and salts and mixtures thereof.

Suitably, the α-hydroxy acid is glycolic acid. In an embodiment, the glycolic acid is present in an amount from about 1% to about 10% by weight, or about 4% to about 10% by weight. According to another embodiment, the α-hydroxy acid is lactic acid.

Exemplary β-hydroxy acids include, but are not limited to, salicylic acid, β-hydroxybutanoic acid, tropic acid and trethocanic acid, and salts and mixtures thereof.

Suitably, the β-hydroxy acid is salicylic acid.

In an embodiment, the kerolytic acid is present in an amount from about 0.1% to about 10% by weight.

Exemplary corticosteroids include, but are not limited to, alclometasone dipropionate, amcinonide, beclomethasone dipropionate, betamethasone benzoate, betamethasone dipropionate, betamethasone valerate, budesonide, clobetasol propionate, clobetasone butyrate, cortisone acetate, desonide, desoximetasone, diflorasone diacetate, diflucortolone valerate, fluclorolone acetonide, flumethasone pivalate, fluocinolone acetonide, fluocinonide, fluocortin butyl, fluocortolone, fluprednidene acetate, flurandrenolide, flurandrenolone, fluticasone propionate, halcinonide, halobetasol propionate, hydrocortisone, hydrocortisone acetate, hydrocortisone butyrate, hydrocortisone propionate, hydrocortisone valerate, methylprednisolone acetate, mometasone furoate, pramoxine hydrochloride, prednisone acetate, prednisone valerate, triamcinolone acetonide, prednicarbate, salts thereof, and mixtures thereof. Suitably, the corticosteroid is present in an amount from about 0.01% to about 2% by weight.

Exemplary antihistamines include, but are not limited to, cetirizine, vapitadine, diphenhydramine, triprolidine, pyrilamine, chlorcyclizine, promethazine, carbinoxamine, tripelennamine, brompheniramine, hydroxyzine, terfenadine, chlorpheniramine, salts thereof, and mixtures thereof.

Exemplary antiseptics include, but are not limited to, hydrogen peroxide, chlorhexidine, cetrimide, povidone iodine, silver sulfadiazine, triclosan, salts thereof, and mixtures thereof.

Exemplary antibacterial agents include, but are not limited to, gentamicin, neomycin, streptomycin, cefpodoxime proxetil, clindamycin, lincomycin, erythromycin, bacitracin, gramicidin(s), vancomycin, doxycycline, minocycline, oxytetracycline, tetracycline, fosfomycin, fusidic acid, mupirocin, sulfacetamide, metronidazole, benzoyl peroxide and dapsone, salts thereof, and mixtures thereof. Suitably, the antibacterial agent is present in an amount from about 0.1% to about 10% by weight.

In an embodiment, the antibacterial agent is clindamycin or a salt thereof, such as clindamycin phosphate. According to an alternative embodiment, the antibacterial agent is metronidazole. In another embodiment, the antibacterial agent is benzoyl peroxide.

Exemplary antifungal agents include, but are not limited to, those selected from the group consisting of echinocandins such as anidulafunin, caspofungin and micafungin; polyenes such as amphotericin B, candicidin, filipin, fungichromin, hachimycin, hamycin, lucensomycin, mepartricin, natamycin, nystatin, pecilocin, perimycin; allylamines such as butenafine, naftifine and terbinafine; imidazoles such as bifonazole, butoconazole, chlormidazole, cloconazole, clotrimazole, econazole, enilconazole, fenticonazole, flutrimazole, isoconazole, ketoconazole, lanoconazole, miconazole, neticonazole, omoconazole, oxiconazole nitrate, sertaconazole, sulconazole and tioconazole; thiocarbamates such as liranaftate, tolciclate, tolindate and tolnafate; triazoles such as albaconazole, pramiconazole, fluconazole, itraconazole, posaconazole, ravuconazole, saperconazole, terconazole and voriconazole; and other antifungal agents such as acrisorcin, amorolfine, biphenamine, bromosalicylchloranilide, buclosamide, calcium propionate, chlorphenesin, ciclopirox, cloxyquin, coparaffinate, exalamide, flucytosine, haloprogin, hexetidine, loflucarban, nifuratel, potassium iodide, propionic acid, pyrithione, salicylanilide, sodium propionate, sulbentine, tenonitrozole, triacetin, undecylenic acid, zinc propionate, griseofulvin, oligomycins, pyrrolnitrin, siccanin, viridian, salts thereof, and mixtures thereof Suitably, the antifungal agent is present in an amount from about 0.1% to about 5% by weight.

Exemplary retinoids include, but are not limited to, tazarotene, tretinoin, isotretinoin, acitretin, etretinate, adapalene, bexarotene, alitretinoin, retinol, retinal, retinyl palmitate, retinyl acetate, retinyl propionate, retinyl linoleate, ethyl 5-(2-(4,4-dimethylthiochroman-6-yl)ethynyl)thiophene-2-carboxylate, 6-(2-4,4-dimethylthiochroman-6-yl)-ethynyl)-3-pyridylmethanol, 6-(2-(4,4-dimethylthiochroman-6-yl)-ethynyl)pyridine-3-carbaldehyde, salts thereof, and mixtures thereof. Suitably, the retinoid is present in an amount from about 0.01% to about 1% by weight.

Exemplary vitamin D analogues include, but are not limited to, calcidiol, calcitriol, calcipotriene, paricalcitol, 22-oxacolcitriol, dihydrotachysterol, calciferol, salts thereof, and mixtures thereof. Suitably, the vitamin D analogue is present in an amount from about 0.001% to about 0.1% by weight.

Exemplary skin-conditioning agents include, but are not limited to, hydrocarbon oils and waxes, silicones, fatty acid derivatives, cholesterol, di- and tri-glycerides, vegetable oils, acetoglyceride esters, alkyl esters, alkenyl esters, lanolin, milk tri-glycerides, wax esters, beeswax, sterols, phospholipids, hyaluronic acid, salts thereof, and mixtures thereof. In an embodiment, the skin-conditioning agent is hyaluronic acid or a salt thereof, such as sodium hyaluronate. Suitably, the skin conditioning agent is present in an amount from about 0.1% to about 20% by weight.

Exemplary nutritional agents include vitamins such as vitamins A, B, C, D, E and K and prodrugs thereof, essential amino acids, essential fats, antioxidants, salts thereof, and mixtures thereof. Suitably, the nutritional agent is present in an amount from about 0.01% to about 2% by weight.

Exemplary sunscreens include, but are not limited to, aminobenzoic acid, avobenzone, bemotrizinol, bisoctrizole, β-carotene, cinoxate, 4-(dimethylamino)benzoic acid, dioxybenzone, drometrizole, ecamsule, ensulizole, ethylhexyl triazone, homosalate, lawsone, menthyl anthranilate, 4-methylbenzylidene camphor, mexenone, octabenzone, octocrylene, octyl methoxycinnamate, octyl salicylate, oxybenzone, sulisobenzone, zinc oxide, titanium dioxide, salts thereof, and mixtures thereof. Suitably, the sunscreen is present in an amount sufficient to provide a sun protection factor of about 4 to about 50.

### Dermatologically Acceptable Excipients

In an embodiment, the present topical aerosol foam compositions further comprise one or more dermatologically acceptable excipients selected from the group consisting of a preservative, an antioxidant and a pH adjusting agent.

### Preservative

Suitably, the preservative is selected from the group consisting of benzyl alcohol, diazolidinyl urea, methyl paraben, ethyl paraben, propyl paraben, butyl paraben, phenoxyethanol, sorbic acid and benzoic acid, salts thereof, and mixtures thereof.

According to a particular embodiment, the preservative is benzyl alcohol.

In an embodiment, the present foam compositions comprise a preservative in an amount from about 0.01% to about 2.5% by weight, from about 0.1% to about 1.5% by weight, or about 1.4% by weight.

### Antioxidant

Suitably, the antioxidant is selected from the group consisting of butylated hydroxytoluene, butylated hydroxyanisole, tocopherol, propyl gallate and vitamin E TPGS, and mixtures thereof. In an embodiment, the present aerosol foam compositions comprise an antioxidant in an amount from about 0.001% to about 1% by weight, from about 0.05% to about 0.5% by weight, or about 0.1% by weight.

### pH Adjusting Agent

In one embodiment, the pH adjusting agent is a base. Suitable pH adjusting bases include bicarbonates, carbonates, and hydroxides such as alkali (e.g. potassium or sodium) or alkaline earth metal hydroxides, as well as transition metal hydroxides. Suitably, the base is potassium hydroxide or sodium hydroxide.

According to another embodiment, the pH adjusting agent is an acid, an acid salt, or a mixture thereof.

In a further embodiment, the pH adjusting agent is a buffer. Suitably, the buffer is selected from the group consisting of citrate/citric acid, acetate/acetic acid, phosphate/phosphoric acid, formate/formic acid, propionate/propionic acid, lactate/lactic acid, carbonate/carbonic acid, ammonium/ammonia, edentate/edetic acid, and mixtures thereof.

In an embodiment, the present foam compositions comprise a pH adjusting agent in an amount sufficient to adjust the pH of the composition to a pH of between about 3.0 to about 9.0. Suitably, the pH of the composition is adjusted to a pH of between about 3.0 to about 4.0. According to an alternative embodiment, the pH is adjusted to a pH between about 5 to about 7.

### The Container

In an embodiment, the topical aerosol foam composition is packaged in a container. Suitably, the container is a pressurized container. The container is selected to provide the composition with a long shelf life and, accordingly, should be chemically inert so as to not interfere with the stability of the composition.

Suitable containers may be made of, for example, steel, aluminum, glass or plastic, and may further employ one or more internal protective linings.

In an embodiment, the aerosol container is a polyamide-imide (PAM) lined aluminium aerosol container.

In one embodiment, the aerosol foam composition comprises: (a) Aloe vera, (b) a surfactant and (c) a propellant.

Suitably the Aloe vera is Aloe vera gel concentrate and the composition further comprises water in an amount from about 40% to about 95% by weight. In an embodiment the surfactant is a non-ionic surfactant. In another embodiment, the non-ionic surfactant is an ethoxylated alcohol.

In another embodiment, the composition further comprises at least one humectant. In another embodiment, the composition further comprises a thickening agent.

In an embodiment, the invention provides a topical aerosol foam composition comprising:
(a) Aloe vera concentrate,
(b) water in an amount from about 40% to about 95% by weight,
(c) at least one humectant in an amount from about 1% to about 20% by weight, and
(d) at least one non-ionic surfactant in an amount from about 0.1% to about 5% by weight.

In another embodiment, the non-ionic surfactant is an ethoxylated alcohol. In yet another embodiment, the composition further comprises a thickening agent. Suitably, in the finished product and aerosol container, there is present a hydrocarbon propellant in an amount from about 2.5% to 10% by weight.

In a further embodiment, the invention provides a topical aerosol foam composition comprising:
(a) Aloe vera concentrate,
(b) a pharmaceutically acceptable active agent,
(c) water in an amount from about 40% to about 95% by weight,
(d) at least one humectant in an amount from about 1% to about 20% by weight, and
(e) at least one non-ionic surfactant in an amount from about 0.1% to about 5% by weight.

Suitably, in an embodiment, the pharmaceutically acceptable active agent is selected from the group consisting of a keratolytic agent, an antibacterial agent and a skin conditioning agent.

Suitably, in the finished product and aerosol container, there is present a hydrocarbon propellant in an amount from about 2.5% to 10% by weight.

In another embodiment, the invention provides a topical aerosol foam composition comprising:
(a) Aloe vera concentrate,
(b) glycolic acid in an amount from about 1% to about 10% by weight,
(c) water in an amount from about 40% to about 95% by weight,
(d) at least one humectant in an amount from about 1% to about 20% by weight, and
(e) at least one non-ionic surfactant in an amount from about 0.1% to about 5% by weight.

In yet another embodiment, the humectant is a mixture of at least 2, or at least 3 or at least 4 humectants. In another embodiment, the humectant is a mixture of glycerin, propylene glycol, sodium PCA and D-panthenol. In an alternative embodiment, the glycerin, propylene glycol, sodium PCA and D-panthenol are present in a ratio of about 1:1:1:1. In another embodiment, the ratio of glycerin, propylene glycol, sodium PCA and D-panthenol is about 1:1:1.4:1.

Suitably, in the finished product and aerosol container, there is present a hydrocarbon propellant in an amount from about 2.5% to 10% by weight.

### Methods of Treatment

The present invention also provides a method of moisturising the skin of a subject in need thereof, comprising topically applying an aerosol foam composition as hereinbefore described. Such moisturization is particularly suitable for the treatment of dry skin.

Furthermore, where the present compositions further comprise a pharmaceutically active agent, they may be used in the treatment of a disease, disorder or condition of the skin (in addition to moisturizing the skin).

Suitably, the disease, disorder or condition of the skin is selected from the group consisting of acne, scars, rosacea, microbial infections, and dermatitis such as psoriasis, seborrhoeic dermatitis, childhood eczema and allergic contact dermatitis. Where the present compositions comprise a keratolytic agent as the active agent, such as a α-hydroxy acid or a β-hydroxy acid, they may be used to gently exfoliate the skin and thus restore or rejuvenate the skin.

In a particular embodiment, the disease, disorder or condition of the skin is acne. In an alternative embodiment, the disease, disorder or condition of the skin is rosacea.

In a further embodiment, the present compositions may be used as part of a regimen for the treatment of a disease, disorder or condition of the skin. In particular, the present compositions may be used in combination with a separate pharmaceutical dosage form. According to an embodiment, the separate pharmaceutical dosage form is a topical preparation. In an alternative embodiment, the separate pharmaceutical dosage form is an oral preparation.

According to an embodiment, the present compositions may be used as part of a regimen for the treatment of acne, where the present compositions are used in combination with a separate topical pharmaceutical dosage form comprising an active ingredient selected from the group consisting of an antibacterial, benzoyl peroxide, salicylic acid and a retinoid, and mixtures thereof.

Suitably, the antibacterial is present in the separate topical pharmaceutical dosage form in an amount from about 0.1 to about 10% by weight. Suitably, the benzoyl peroxide is present in an amount from about 1% to about 10% by weight. Suitably, the salicylic acid is present in an amount from about 0.1% to about 5% by weight. Suitably, the retinoid is present in an amount from about 0.01% to about 1% by weight.

In an embodiment, the separate pharmaceutical dosage form is an aerosol foam. According to a particular embodiment, the separate pharmaceutical dosage form is an aerosol foam comprising benzoyl peroxide. Suitably, the aerosol foam comprising benzoyl peroxide is an aqueous foam. In an embodiment, the aqueous aerosol foam comprises: an aqueous phase comprising water, benzoyl peroxide present in an amount from about 1% to about 10% by weight suspended in the aqueous phase, a thickening agent, and an aerosol propellant. An exemplary benzoyl peroxide aqueous foam (as a separate dosage form) is described is Example 5.

In a further embodiment, the separate dosage form is a topical aerosol foam composition comprising salicylic acid. Suitably, the aerosol foam comprises salicylic acid, water, ethanol in an amount from about 5% to 40% by weight, wax, surfactant and an aerosol propellant.

### Definitions

The phrases an “effective amount”, “an amount effective to”, a “therapeutically effective amount” or a “cosmetically effective amount” are used herein to refer to an amount of the composition sufficient to have a therapeutic or cosmetic effect upon administration. Effective amounts will vary with the particular condition or conditions being treated, the severity of the condition, the duration of the treatment, and the specific components of the composition.

The terms “administering” and “administration” are used herein to mean any method which in sound cosmetic or medical practice delivers the composition to a subject in such a manner as to provide the desired cosmetic or therapeutic effect.

The terms “treatment” or “treating” of a skin disease, disorder or condition encompasses alleviation of at least one symptom thereof, a reduction in the severity thereof, or the delay, prevention or inhibition of the progression thereof. Treatment need not mean that the disorder is totally cured. A useful composition herein need only to reduce the severity of the disorder, reduce the severity of symptoms associated therewith, provide improvement to a patient's quality of life, or delay, prevent or inhibit the onset of the disorder.

The term “salt thereof” refers to salts that are pharmaceutically or cosmetically acceptable. Such salts include: (1) acid addition salts, formed with acids such as, for example, acetic acid, benzoic acid, citric acid, gluconic acid, glutamic acid, glutaric acid, glycolic acid, hydrochloric acid, lactic acid, maleic acid, malic acid, malonic acid, mandelic acid, phosphoric acid, propionic acid, sorbic acid, succinic acid, sulfuric acid, tartaric acid, naturally and synthetically derived amino acids, and mixtures thereof; or (2) salts formed when an acidic proton present in the parent compound is either (i) replaced by a metal ion e.g. an alkali metal ion, an alkaline earth metal ion or an aluminium ion; or (ii) protonates an organic base such as, for example, ethanolamine, diethanolamine, triethanolamine, tromethamine and N-methylglucamine.

Any concentration range, percentage range or ratio range recited herein is to be understood to include concentrations, percentages or ratios of any integer within that range and fractions thereof, such as one tenth and one hundredth of an integer, unless otherwise indicated.

It should be understood that the terms “a” and “an” as used herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

Throughout the application, descriptions of various embodiments use “comprising” language, however in some specific instances, an embodiment can alternatively be described using the language “consisting essentially of or “consisting of”.

All numbers expressing quantities, percentages or proportions, and other numerical values used in the specification and claims, are to be understood as being modified in all instances by the term “about.”

“Substantially devoid” of a specified component refers to a composition with less than about 1% by weight of the specified component.

Other terms used herein are intended to be defined by their well known meanings in the art.

### EXAMPLES

### Example 1

### Revitalizing Toner Foam

| Item | Ingredient | % by weight |
|---|---|---|
| 1 | water | 82.38 |
| 2 | potassium hydroxide | 1.82 |
| 3 | glycolic acid (70% solution) | 8.50 |
| 4 | hydroxyl ethyl cellulose | 0.20 |
| 5 | decyl polyglucoside | 1.00 |
| 6 | propylene glycol | 0.50 |
| 7 | glycerin | 0.50 |
| 8 | sodium PCA (50% solution) | 0.70 |
| 9 | D-panthenol (75% solution) | 0.50 |
| 10 | benzyl alcohol | 1.40 |
| 11 | 40× Aloe vera gel | 2.50 |
| | TOTAL | 100.00 |
| 12 | Propellant AP 70 | 5.00 |
| | (propane, butane & isobutane) | |

### Example 2

### Revitalizing Toner Foam

| Item | Ingredient | % by weight |
|---|---|---|
| 1 | water | 82.38 |
| 2 | potassium hydroxide | 1.82 |
| 3 | glycolic acid (70% solution) | 8.50 |
| 4 | hydroxyl ethyl cellulose | 0.20 |
| 5 | ceteareth-20 | 1.00 |
| 6 | propylene glycol | 0.50 |
| 7 | glycerin | 0.50 |
| 8 | sodium PCA (50% solution) | 0.70 |
| 9 | D-panthenol (75% solution) | 0.50 |
| 10 | benzyl alcohol | 1.40 |
| 11 | 40× Aloe vera gel | 2.50 |
| | TOTAL | 100.00 |
| 12 | Propellant AP 70 | 4.50 |
| | (propane, butane & isobutane) | |

### Example 3

### Method of Preparation

Ingredients 1 to 11 were successively added to a mixing vessel, while stirring, as follows:
(i) water was added to the mixing vessel,
(ii) potassium hydroxide was added to the water and stirred until dissolved,
(iii) glycolic acid solution was slowly added whilst stirring, the contents of the vessel were allowed to cool below 30° C. whilst stirring for at least 1 hour (to facilitate de-oligomerisation of the glycolic acid),
(iv) while stirring, hydroxyethyl cellulose was slowly added to the vessel and stirred until dissolved,
(v) with gentle stirring, decyl polyglucoside or ceteareth-20 was added and stirred until dissolved,
(vi) propylene glycol, glycerin, sodium PCA and D-panthenol were added with stirring,
(vii) benzyl alcohol was then added and mixed until homogenous, and
(viii) the Aloe vera was added. The final pH was between 3 and 4.

The final reaction mixture (aerosol foam base) was a single phase aqueous composition. The aerosol base was subsequently added to an aerosol can, crimped and filled with hydrocarbon propellant to give the final aerosol product. Following actuation of the valve, an elegant aerosol foam was dispensed, which was suitable for application to the face.

The final aerosol foam had a stable pH when subjected to storage at 40° C. for six months.

### Example 4

### Therapeutic and Cosmetic Toner Foams

The following aerosol foams comprising Aloe vera further exemplify the present invention.

### Example 4(a)

### Revitalising Aloe Vera Foam with Salicylic Acid

Aloe vera foam with salicylic acid

| Ingredient | % by weight |
|---|---|
| water, purified | 85.00 |
| sodium hydroxide (97%) | 0.15 |
| salicylic acid | 0.50 |
| hydroxyethyl cellulose | 0.20 |
| decyl polyglucoside | 1.00 |
| glycerin | 2.00 |
| phenoxyethanol | 0.75 |
| 10% citric acid (aq) or | to pH 6-7 |
| 10% sodium hydroxide (aq) | |
| 40× Aloe Vera gel | 2.50 |
| water, purified | to 100% |
| TOTAL | 100.00 |
| hydrocarbon propellant AP70 | Add 5.0 g of |
| (propane, butane and | propellant |
| isobutane) | per 100.0 g of |
| | aerosol |
| | base (above). |

### Example 4(b)

### Hydrating Aloe Vera Foam with Sodium Hyaluronate

Aloe vera foam with sodium hyaluronate

| Ingredient | % by weight |
|---|---|
| water, purified | 85.00 |
| sodium hyaluronate | 0.20 |
| decyl polyglucoside | 1.00 |
| glycerin | 1.00 |
| phenoxyethanol | 0.75 |
| 10% citric acid (aq) or | to pH 5-6 |
| 10% sodium hydroxide (aq) | |
| 200× Aloe Vera gel | 0.50 |
| water, purified | to 100% |
| TOTAL | 100.00 |
| hydrocarbon propellant AP70 | Add 5.0 g of propellant |
| (propane, butane and | per 100.0 g of aerosol |
| isobutane) | base (above). |

### Example 4(c)

### Therapeutic Aloe Vera Foam with Metronidazole

Aloe vera foam with metronidazole

| Ingredient | % by weight |
|---|---|
| water, purified | 93.00 |
| nicotinamide | 1.00 |
| beta-cyclodextrin | 0.50 |
| metronidazole | 1.00 |
| ceteareth-20 | 0.50 |
| glycerin | 2.50 |
| benzyl alcohol | 1.00 |
| 200× Aloe Vera gel | 0.50 |
| TOTAL | 100.00 |
| hydrocarbon propellant AP70 | Add 5.0 g of propellant |
| (propane, butane and | per 100.0 g of aerosol |
| isobutane) | base (above). |

### Example 4(d)

### Therapeutic Aloe Vera Foam with Clindamycin Phosphate

Aloe vera foam with clindamycin phosphate

| Ingredient | % by weight |
|---|---|
| water, purified | 85.00 |
| clindamycin phosphate | 1.25 |
| ceteareth-20 | 0.50 |
| propylene glycol | 1.00 |
| benzyl alcohol | 1.00 |
| 200× Aloe Vera gel | 0.50 |
| 1% potassium hydroxide (aq) | to pH 4.0-4.5 |
| water, purified | to 100% |
| TOTAL | 100.00 |
| hydrocarbon propellant AP70 | Add 5.0 g of propellant |
| (propane, butane and | per 100.0 g of aerosol |
| isobutane) | base (above). |

### Example 5

### Benzoyl Peroxide Aqueous Foam as a Separate Dosage Form for Use in Conjunction with an Aloe Vera Foam

Benzoyl peroxide aqueous foam

| Ingredient | % by weight |
|---|---|
| water | 93.065 |
| ceteareth-20 | 1 |
| citric acid | 0.15 |
| sodium citrate | 0.35 |
| benzoyl peroxide | 3.335 |
| disodium EDTA | 0.1 |
| xanthan gum | 1 |
| benzyl alcohol | 1 |
| TOTAL | 100.00 |
| hydrocarbon propellant | Add 5.0 g of propellant |
| AP70 (propane, butane and | per 95 g of aerosol |
| isobutane) | base (as above). |

Although specific embodiments of the invention have been described above, it will be clear to the skilled addressee that the invention is not restricted to these particular embodiments, and that variations may be made without departing from the scope of the present invention.

1-23. (canceled)

24. A topical aerosol foam composition comprising: (a) Aloe vera, (b) a surfactant and (c) a hydrocarbon propellant, and (d) a keratolytic agent.

25. The composition according to claim 24, wherein the Aloe vera is Aloe barbadensis leaf juice.

26. The composition according to claim 24, wherein the Aloe vera is Aloe vera gel concentrate and the composition further comprises water in an amount from about 40% to about 95% by weight.

27. The composition according to claim 26, wherein the composition comprises water in an amount from about 70% to about 95% by weight.

28. The composition according to claim 24, wherein the surfactant is a non-ionic surfactant.

29. The composition according to claim 28, wherein the non-ionic surfactant is an ethoxylated alcohol.

30. The composition according to claim 29, wherein the ethoxylated alcohol is selected from the group consisting of ceteareth-6, ceteareth-12, ceteareth-20, ceteareth-25, ceteth-10, ceteth-20, laureth-4, laureth-5, laureth-9, laureth-10, laureth-12, laureth-15, laureth-20, laureth-23, oleth-10, oleth-20, steareth-10, steareth-20 and steareth-100, and mixtures thereof

31. The composition according to claim 30, wherein the ethoxylated alcohol is ceteareth-20.

32. The composition according to claim 24, wherein the hydrocarbon propellant is a mixture of propane, n-butane, and isobutane.

33. The composition according to 24, further comprising a humectant.

34. The composition according to claim 24, further comprising a thickening agent.

35. The composition according to claim 24, wherein the keratolytic agent is an acid selected from the group consisting of an α-hydroxy acid and a β-hydroxy acid, and mixtures thereof.

36. The composition according to claim 35, wherein the keratolytic agent is a α-hydroxy acid.

37. The composition according to claim 36, wherein the α-hydroxy acid is glycolic acid.

38. The composition according to claim 33, wherein the keratolytic agent is a α-hydroxy acid, and the non-ionic surfactant is an ethoxylated alcohol.

39. A topical aerosol foam composition comprising:
(a) Aloe vera concentrate;
(b) glycolic acid in an amount from about 1% to about 10% by weight;
(c) water in an amount from about 40% to about 95% by weight;
(d) at least one humectant in an amount from about 1% to about 20% by weight;
(e) at least one non-ionic surfactant in an amount from about 0.1% to about 5% by weight; and
(f) a hydrocarbon propellant in an amount from about 2.5% to 10% by weight.

40. A method of moisturizing the skin, the method comprising topically administering to a subject in need thereof, a cosmetically effective amount of an aerosol foam composition according to claim 24.

41. A method of treating a disease, disorder or condition of the skin, the method comprising topically administering to a subject in need thereof, a therapeutically effective amount of an aerosol foam composition according to claim 24.
